(19) United States
(12) Patent Application Publication
(10) Pub. No.: US 2023/0380529 A1
(43) Pub. Date: Nov. 30, 2023

(54) Wristband Apparatus

(71) Applicant: Rose Anne Rodriguez, Ketchikan, AK (US)

(72) Inventor: Rose Anne Rodriguez, Ketchikan, AK (US)

(21) Appl. No.: 17/752,124

(22) Filed: May 24, 2022

(51) Int. Cl. A41D 20/00; A41D 27/20; A41D 31/12

(52) U.S. Cl. CPC A41D 20/00; A41D 27/20; A41D 31/125; A61F 2013/530364

(57) ABSTRACT

The present disclosure provides a wristband apparatus having a plurality of high capacity absorbent pockets. Elastic bands and seams between the pockets maintain the structure of the wristband apparatus even when large amounts of fluid have been absorbed. Since large amounts of fluid can be absorbed, the wristband apparatus can double as an exercise weight which puts no strain on the wrist, is comfortable to wear, and which simultaneously cools the body. The wristband apparatus can also be used as a normal exercise wristband, and even to prevent excess fluids from running down a user's arms when they wash in a sink basin.

### FIELD OF INVENTION

The present invention relates to a wristband apparatus, and more particularly to a wristband comprising a plurality of absorbent pockets configured to absorb high levels of fluid.

### BACKGROUND

Wristbands are known in the art most commonly as exercise apparatus for supporting a user's wrist during exercise by applying moderate pressure and absorbing some of the force of external impacts to the wrists, for instance if the user is recovering from injury, and for absorbing bodily fluids such as excess sweat.

They generally consist of either a flexible strap with connectible ends or a flexible and elastic fabric loop or tube which wraps around a user's wrist. The strap variety are less convenient to use since they require a user to secure the ends together about one wrist using only their opposing hand.

Both varieties are usually of uniform thickness and configured to absorb moderate levels of fluid so that a user can wipe the sweat from their brow and keep their arms from getting too slick, but are generally limited in their fluid absorption capacity in favour of maintaining a compact design that does not expand about the wrists too much.

In recent years there has been a dramatic increase in promoting health and well being by regular exercise, with an emphasis on strength training. This type of aerobic exercise often puts large pressures on the wrists by use of dumbbells and other handheld weights, limiting the range of movements a user can safely make with them. There are other solutions, such as sandbag-type wrist weights, which comprise weighted pockets which wrap around a user's wrist and thus avoid the same strain and risk of injury, however these quickly become uncomfortable to wear as the body produces sweat and their body temperature increases.

It is within this context that the present invention is provided.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present disclosure provides a wristband apparatus having a plurality of high capacity absorbent pockets. Elastic bands and seams between the pockets maintain the structure of the wristband apparatus even when large amounts of fluid have been absorbed. Since large amounts of fluid can be absorbed, the wristband apparatus can double as an exercise weight which puts no strain on the wrist, is comfortable to wear, and which simultaneously cools the body. The wristband apparatus can also be used as a normal exercise wristband, and even to prevent excess fluids from running down a user's arms when they wash in a sink basin.

Thus, according to one aspect of the present disclosure there is provided a wrist band, comprising: a tubular band body having a central axis oriented along a first direction, the band body being formed of a flexible outer fabric layer and an inner layer formed of one or more absorbent articles, the outer layer being structured so as to encapsulate the one or more absorbent articles of the inner layer in a plurality of pockets arranged about the central axis, and wherein the outer layer and inner layer are secured together by a plurality of seams oriented in a direction parallel to the central axis; and one or more elastic bands encompassing the band body about the central axis so as to compress the band body against the wrist of a wearer.

In some embodiments, a separate absorbent article is provided for and encapsulated within each pocket formed by the outer layer fabric. In other embodiments, the inner layer is comprised of a single absorbent article formed into pockets.

In some embodiments, the outer layer is formed so as to accommodate volumetric expansion of the one or more absorbent articles within the plurality of pockets by a factor of 2 or more.

In some embodiments, the one or more absorbent articles are formed of a porous cloth. In other embodiments, the one or more absorbent articles are formed of flannel or towel fabric.

In some embodiments, the outer layer is formed of spandex. In other embodiments, the outer layer is formed of a natural bamboo moisture wicking fabric.

In some embodiments, the one or more elastic bands are sewn onto the band body.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

Various embodiments of the invention are disclosed in the following detailed description and accompanying drawings.

FIG. 1 is a perspective view illustrating a pair of wristbands according to a first embodiment of the present disclosure being worn by a user;

FIG. 2 is a perspective view illustrating the pair of wristbands according to the first embodiment in use;

FIG. 3 is a cross-sectional view illustrating the wrist band shown in FIG. 1.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

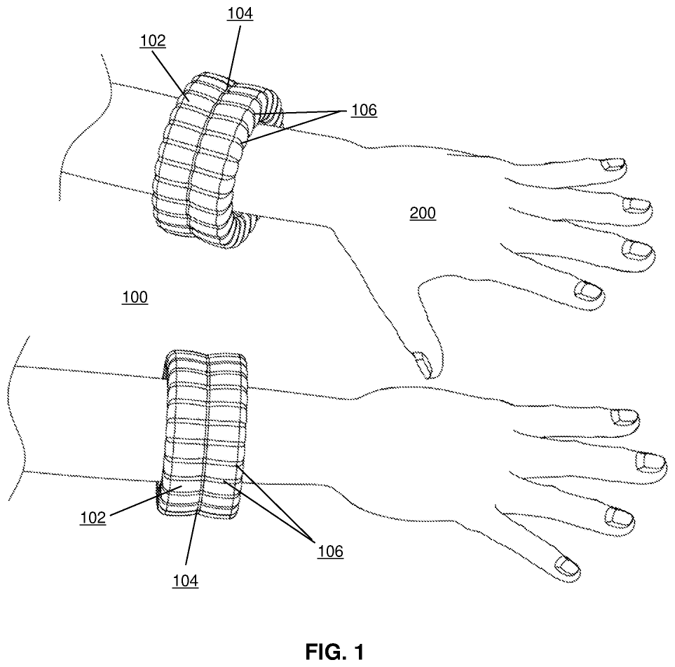

Referring to FIG. 1, a perspective view of a pair of wristband apparatus 100 according to one example configuration is shown worn on the wrists of a user 200.

As can be seen, the wristband apparatus 100 are each formed of a tubular band body 102 which a user can easily insert their hands through due to it being formed of elastic materials. The band body is held in place and applies pressure to a user's wrist as a result of one or more elastic bands 104 disposed around the outer layer. The elastic bands should be of high strength to ensure that expansion of the wristband due to absorption of fluid does not cause the wristbands to become loose.

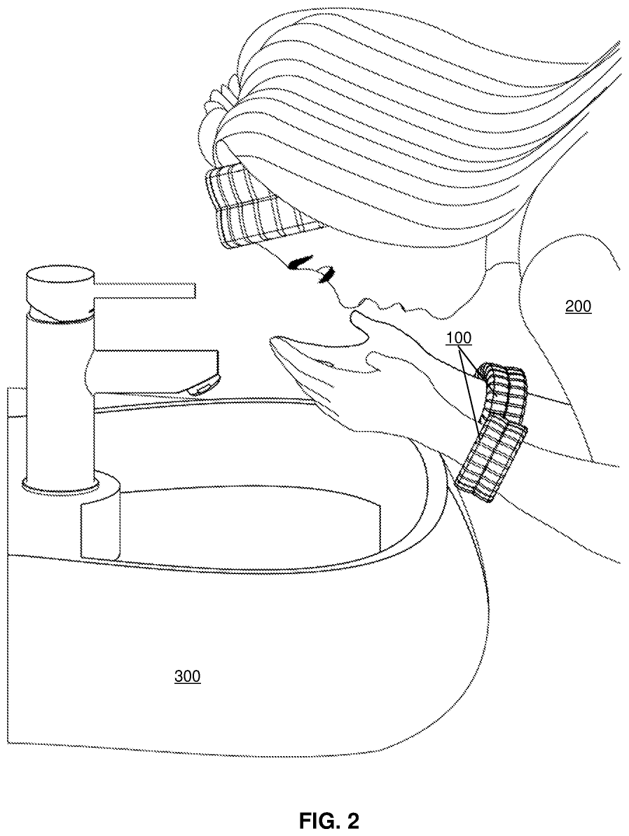

The band body 102 is formed of an outer fabric layer, the only layer visible in FIG. 1 and FIG. 2, and an inner layer comprised of one or more absorbent articles such as flannel or cloth elements. The band body 102 is held together and also divided into a plurality of pockets by a plurality of seams running in a longitudinal direction parallel to a central axis of the tubular body and parallel to a user's radius/ulna when the wristband is worn.

These seams 106 are a key feature of the wristband apparatus, as they facilitate large amounts of volumetric expansion of the absorbent article within each pocket while maintaining the structural integrity of the band. Wristbands of the prior art are limited in the amount of liquid they are designed to absorb, because after a certain amount of expansion the bands become heavy and impede some types of aerobic exercise. The inventor of the present disclosure has recognised that this can in fact be advantageous in strengthening exercises, adding exercise weight functionality to the wristband while maintaining a firm and comfortable placement on the wrist that will continue to absorb excess bodily fluids and also regulate the user's body temperature.

The wristband apparatus 100 can also double up as an accessory for preventing water from running down a user's forearms when they wash their face.

Referring to FIG. 2, a user 200 is shown washing in a basin 300 while wearing a pair of wristband apparatus as shown in the example of FIG. 1. Often, when washing in this way, a user risks excess water running down their forearms as they raise their hands to their face, which can feel unpleasant and cause clothing to get wet. The wristbands 100 of the present example can help prevent this due to their high capacity for absorption.

As shown, the wristband apparatus may be paired with a spa headband.

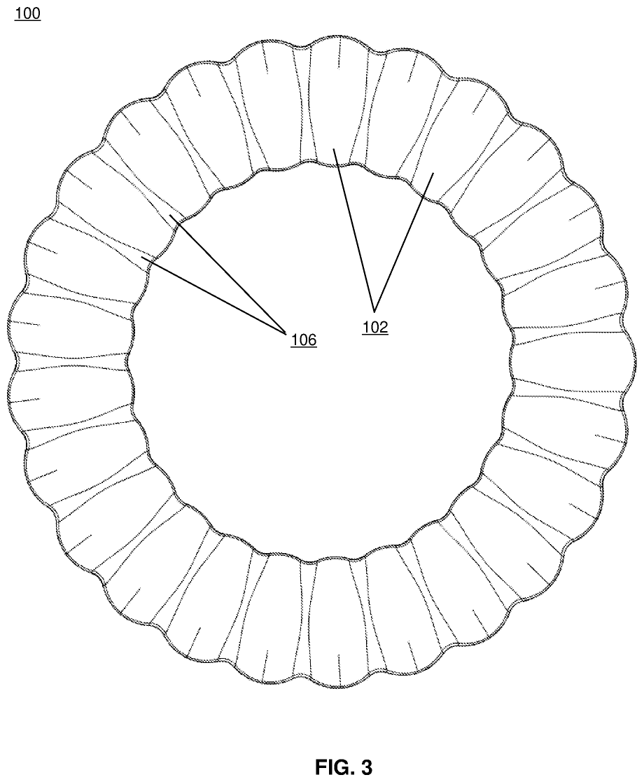

Referring to FIG. 3, a cross-sectional view of an example wristband 100 according to the present disclosure is shown. The effects of the seams 106 compressing the inner and outer fabric layers into pockets is more clear form the perspective.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the wristband apparatus have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, the inventive subject matter lies in less than all features of a single disclosed embodiment. Thus, the following claims are hereby incorporated into the Detailed Description, with each claim standing on its own as a separately claimed subject matter.

What is claimed is:

1. A wristband apparatus, comprising:
a tubular band body having a central axis oriented along a first direction, the band body being formed of a flexible outer fabric layer and an inner layer formed of one or more absorbent articles, the outer layer being structured so as to encapsulate the one or more absorbent articles of the inner layer in a plurality of pockets arranged about the central axis, and wherein the outer layer and inner layer are secured together by a plurality of seams oriented in a direction parallel to the central axis; and
one or more elastic bands encompassing the band body about the central axis so as to compress the band body against the wrist of a wearer.

2. A wristband apparatus according to claim 1, wherein a separate absorbent article is provided for and encapsulated within each pocket formed by the outer layer fabric.

3. A wristband apparatus according to claim 1, wherein the inner layer is comprised of a single absorbent article formed into pockets.

4. A wristband apparatus according to claim 1, wherein the outer layer is formed so as to accommodate volumetric expansion of the one or more absorbent articles within the plurality of pockets by a factor of 2 or more.

5. A wristband apparatus according to claim 1, wherein the one or more absorbent articles are formed of a porous cloth.

6. A wristband apparatus according to claim 1, wherein the one or more absorbent articles are formed of flannel or towel fabric.

7. A wristband apparatus according to claim 1, wherein the outer layer is formed of spandex.

8. A wristband apparatus according to claim 1, wherein the outer layer is formed of a natural bamboo moisture wicking fabric.

9. A wristband apparatus according to claim 1, wherein the one or more elastic bands are sewn onto the band body.
